(19) United States
(12) Patent Application Publication
(10) Pub. No.: US 2008/0132860 A1
(43) Pub. Date: Jun. 5, 2008

(54) APPARATUS, SYSTEM AND METHOD FOR DETECTING SURGICAL SPONGES IN SURGICAL PATIENTS AND SURGICAL DRAPES

(76) Inventors: William Smith, Bloomington, IL (US); Nelson Slavik, Niles, MI (US); David Lundquist, Stony Brook, NY (US)

Correspondence Address: MICHAEL BERNS;MALONEY, PARKINSON AND BERNS, 135 W MAIN STREET, URBANA, IL, 61801, US

(21) Appl. No.: 11/949,630

(22) Filed: Dec. 3, 2007

Related U.S. Application Data
(60) Provisional application No. 60/868,192, filed on Dec. 1, 2006.

(51) Int. Cl. A61F 13/15

(52) U.S. Cl. 604/362

(57) ABSTRACT

The Sponge Detection System invention consists of surgical sponges or devices with integrated metallic strips that can be easily detected by the invention s specially designed electromagnetic wand detection scanner. The surgical sponges will be used by hospitals in an effort to minimize the potential for sponges that are left behind during surgeries.

### CROSS-REFERENCE TO RELATED APPLICATION

This application claims the benefit of U.S. Provisional Application 60/868,192, filed Dec. 1, 2006, which is incorporated herein by reference.

### BACKGROUND OF THE INVENTION

The present invention relates to a system and method of detecting surgical sponges that have been unaccounted for after patient surgery, where there is a distinct possibility that the sponges have been retained in the patient or in the surgical drapes used during surgery. More specifically the invention relates to surgical sponges that are commonly comprised of radio opaque (x-ray detectable) PVC barium sulfate ribbons or strings that have been woven or sewn into the surgical sponge, however in the context of this invention an elongated thin magnetic amorphous metal wire or filament (of various dimension in width and thickness) has been embedded or co-extruded within the PVC barium sulfate ribbon or string exhibiting a highly non-linear magnetic field response enabling a wand scanner to be passed over the patient thereby detecting the sponge

### SUMMARY OF THE INVENTION

The Sponge Detection System invention consists of surgical sponges or devices with integrated metallic strips that can be easily detected by the invention s specially designed electromagnetic wand detection scanner. The surgical sponges will be used by hospitals in an effort to minimize the potential for sponges that are left behind during surgeries.

### DESCRIPTION OF THE DRAWINGS

FIG. 1 is an illustration of an embodiment of the invention as a plastic ribbon inserted within a surgical sponge.

FIG. 2 is an illustration of an embodiment of the invention showing a detail of the plastic ribbon having a magnetic amorphous wire inside.

FIG. 3 is an illustration of an embodiment of the invention showing a plastic string woven into a gauze surgical sponge.

FIG. 4 is an illustration of a plastic string having a magnetic amorphous wire inside.

FIG. 5 is an illustration of an extrusion process to insert a magnetic amorphous wire into a polyvinyl cholride barium sulfate ribbon.

FIG. 6 is an illustration of an embodiment of a detector device in a circular model that may be used with two hands.

FIG. 7 is an illustration of an embodiment of a detector device as shown in FIG. 6, in an oblique view.

### DETAILED DESCRIPTION OF THE INVENTION

There is an abundance of prior art relating to the marking or tagging of surgical sponges and instruments using either bar-coding, electromagnetic or radio frequency identification technology, however there is no prior art relating specifically to imbedding or co-extruding an amorphous wire into the PVC barium sulfate ribbon that is woven or sewn into the sponge and specifically to a self-contained scanner wand detection device that passes over the patient.

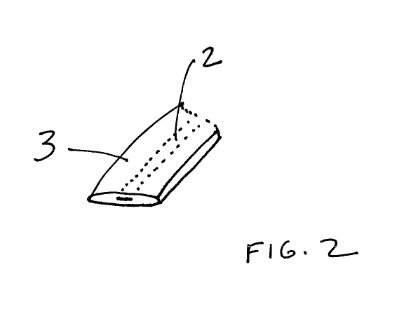

The invention may include the use of a sponge or other medical device that incorporates a ribbon as shown in FIG. 2, showing a barium sulfate ribbon with an amorphous metal wire co-extruded into the ribbon.

Detection of the medical device may be accomplished by a scanner or either a paddle design with a handle for use with one hand, or a circular wheel style shown in FIG. 6 and FIG. 7.

A medical device may be marked with the amorphous wire as described. Detection of that wire and associated medical device may be accomplished by the use of a scanning device as described.

The following describes the detection portion of a complete system for detecting surgical sponges accidentally left inside patients during surgical procedures. The total system is composed of two elements: A standard surgical sponge to which a unique magnetic marker has been attached, and a wand like device that is passed over the patient which will detect sponges to which a magnetic marker has been added.

A number of technologies have been considered as candidates for implementing such a system. The approach described here is based on one commonly used in the anti-theft or EAS (electronic article surveillance) industry, This technique uses as tags elongated strips of metallic ribbon that exhibit a highly non-linear magnetic field response. When placed in a low frequency AC magnetic field, the magnetic domains of these materials will change polarity suddenly to follow the bias of the externally applied field. This sudden reversal of magnetization near each zero crossing causes the tag to emit a pulse like response rich in harmonics and synchronous with the applied field. The unique characteristics of this emitted field can be detected by a receiver and distinguished from environmental noise and signals emitted by other ferromagnetic objects.

While commercial systems to detect such tags are common in libraries, video rental stores, and even supermarkets in many parts of the world, an implementation suitable to detect tags embedded in surgical sponges in an operating room has very different design criteria. The following is a discussion of the challenges in creating such a detection wand and the methods used to implement a successful system. Examples of potential wand (scanner) designs are enclosed in Exhibit “B”.

### AC Field Excitation Coil Design.

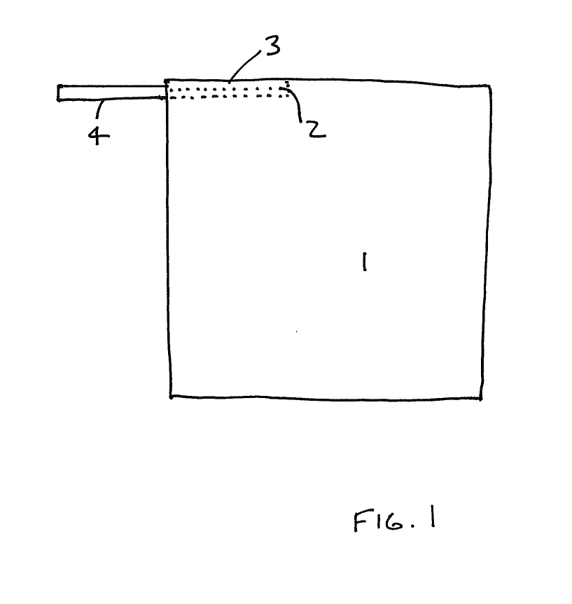

An AC magnetic field sufficiently strong must be provided in the entire three-dimensional space where tags are to be detected. It is desirable to do this with the smallest and lighted possible means so that it may be easily positioned over the patient in the area where sponges are likely to have been left. While small size is preferred, it can be seen from FIG. 1 below that as we move away from a simple coil of wire through which a current has been passed, the field strength begins to fall off more rapidly.

From FIG. 1 we conclude that the larger the coil, the further the distance achieved before the magnetic field begins to drop rapidly. Our findings are that a coil of at least 12 inches in diameter is required to achieve field strengths necessary to excite a tagat the desired range. Our system has been designed with an excitation coil of 15 inches diameter to provide design margin in achieving range sufficient to detect sponges at a depth deemed sufficient for the vast majority of procedures.

Another aspect of excitation coil design that will influence system ease of use is the selection of the conductor material. Copper is commonly used in coils due to its good conductivity and the fact that it is easy to connect to using soldered connections. While copper is the best material from a volume standpoint it is not optimal from a weight standpoint. For example for a given wire diameter, aluminum has only 62% of the conductivity of the copper. More importantly though the aluminum wire only weighs 30% of the copper one. Thus even if we make the aluminum wire thicker by a factor of 1.62 to match the conductivity of the copper wire, it is still only 1.62×30% or just under half the weight of the copper one. For a hand held unit where minimizing weight is important, aluminum wire is the preferred material for the drive coil and has bee selected for our system.

Lastly the shape of the excitation coil must be considered. Physics dictates that to produce a given field at a certain distance with the least power, the optimal shape for a wound coil is a circle. Our system will thus use simple circular coil shape to produce the AC drive field.

Frequency of the AC drive signal is another system design consideration. Some anti-theft applications use drive signals as low as 70 Hz, while others using essentially the same tags operate as high as 20 KHz or 20,000 Hz. In general it is desirable to increase the drive frequency as this produces mord tag signal power due to the increased number of zero crossings per second. Arguing against this is the increased sensitivity to environmental noise at higher frequencies, and the loss of distinction between tags and other ferromagnetic items at the higher frequencies. Experience has shown that a reasonable compromise is to operate at approximately 1.0 KHz although system can be made to work reasonably well over the full range mentioned previously.

### Driving the Excitation Coil

The first important aspect of coil drive is the requirement to resonate the coil with a capacitor so that the resonant frequency is set at the desired operating frequency. The need to resonant coil is due to its natural inductive reactance. If not resonated, a large amount of power is wasted driving the reactive portion of the coil impedance. The resonating capacitor cancels this resistance at one frequency and makes the coil appear to the systems a simple resistor. While there are a number of circuit configurations that may be used, the basic choice is between series or parallel resonant as shown below:

We have chosen a direct series resonant configuration as it offers safety in the resonant impedance increases, and thus current decreases if there is any fault or mis-tuning of the circuit. It also offers additional filtering of the excitation signal as voltages at frequencies other than the desired frequency see a higher impedance than the desired signal. Wire gauge and number of turns is chosen to maximize the field for a given current and provide a DC resistance of between 4 and 10 Ohms.

Power required to drive the excitation coil is somewhat less than that required when this technology is used in EAS applications. In those systems power levels are generally between 10 W and 60 W. In our preferred embodiment system the applied power will be between 10 and 20 Watts. These power levels are proportionally less than the EAS systems by the approximate ratio of detection zone covered. Class AB Audio amplifier integrated circuits or similar discrete transistor designs have traditionally been used in EAS systems. Recent developments in more efficient and smaller class D switch mode amplifiers make them ideal candidates to drive the excitation coil in our design. One example of such a device is the AD 1994 from Analog Devices, Inc. Other circuitry in the detection wand will provide a 1 KHz signal to drive the amplifier.

### Detection Coil Design.

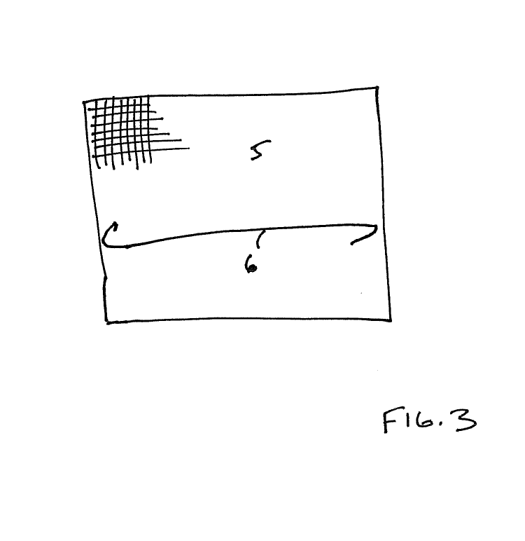

While the excitation coil provides the necessary low frequency AC magnetic field to drive the polarity of the magnetic domains in the tag back and forth, a separate coil is used the detect the resulting harmonic energy emanating from the tag. The need for this separate coil arises from the very large difference in strength of the two fields. The excitation coil may have several amps of current passing through it while the receive field may only induce a few micro amps of current to be detected. If there were no isolation provided it would be very difficult to detect the minuscule tag signal in the presence of the large excitation signal. The most common way to do this is by designing the detection coil so that when it is placed on top of the excitation coil there is a net cancellation of the excitation field in the summation of the signal from the two opposite phased halves of the detection coil. FIG. 3 below shows this coil arrangement and how it produces the excitation field cancellation. Now instead of amps of excitation current, the detection processing circuitry may only see a few milliamps of signal from the excitation field. Processing of the tag signal is now much easier and may be done with greater accuracy and reliability.

### Wand Electronics Overview

The electronics for the wand are contained on a single circuit board (PCB) located in the handle of the unit. Fundamentally there are 3 main functions performed by this PCB.
+Excitation Coil Drive
+Detection Coil Signal Processing
+Power Management

We will review these three units beginning with the simplest one, the excitation coil drive.

### Excitation Coil Drive

The system controller, a single chip micro-controller or DSP provides a nominal 1.0 KHz square wave reference signal when the system is energized. The controller has the ability to adjust this frequency to match the resonant frequency of the excitation coil, resonating capacitor combination to provide optimal drive efficiency.

This frequency reference is fed into a bandpass or lowpass filter that removes the harmonic content from the signal thus changing it into the desired sine wave drive signal.

A gain adjust block then provides a means for the system controller to vary the drive level to the power amplifier.

The excitation coil power amplifier is a class D integrated circuit device capable of providing at least 20 W of drive to the excitation coil that appears as an approximate 4-ohm resistive load at resonance.

Continuous time samples of the excitation drive voltage and current are provided back to the system controller to allow it to perform a number of monitoring and control functions. These include:

Adjusting the drive level to achieve a desired coil current. Adjusting the drive frequency to match the exact resonance frequency of the excitation coil, resonating capacitor combination. Detection of excitation faults due to gross metal loading, or other internal faults.

### Detection Signal Processing.

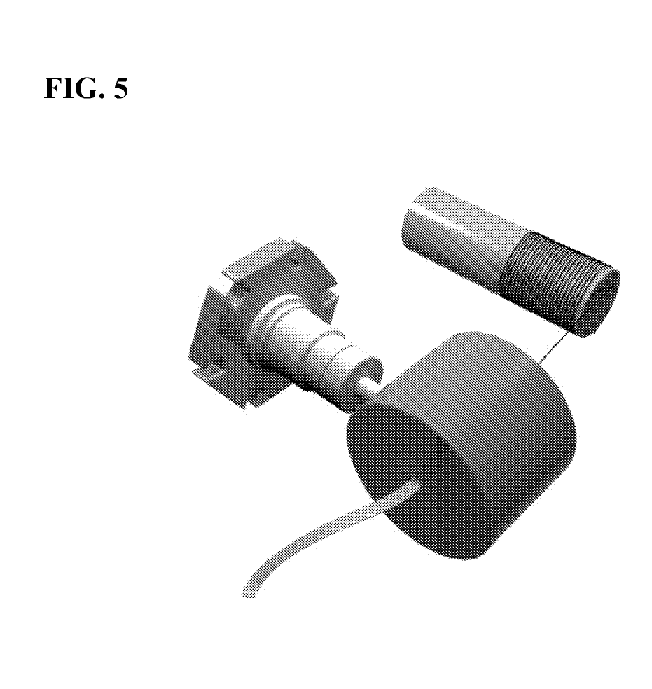

Processing the signal induced in the detection coil is the single most complex part of the system. This is due to many factors which include: The very low level of the induced signal. The presence of interfering signals from nearby electronic equipment, and the need to distinguish the tag signal from that produced by other metallic objects. As such, a combination of analog and digital processing is used to achieve the best performance in both detection rate and rejection of non-tag signals. A block diagram of the receive signal processing chain is shown in FIG. 5 below. A discussion of the individual elements follows:

### Input Pre-amplifier

This block connects directly to the detection coil and provides the initial gain for the tag signal and rejection of some interfering signals. Gain value is typically 20 dB. A low noise Op-Amp is used in a differential configuration to reject common mode interference and contribute as little as possible to the system noise floor. Input referred noise levels below 4nVIVHz will be required for good performance. Common Mode rejection ratios of more than 40 dB will also be provided.

### Excitation Notch Filter

An active twin T notch filter follows the input preamplifier and serves to provide rejection of any remaining excitation field energy not cancelled by the first order rejection of the excitation, detection coil pair. No additional gain is provided here. The notch is sufficiently wide to reject the range of excitation frequencies the controller can generate.

### System Bandpass+Gain Block

Following tire notch filter a number of cascadled highpass and lowpass filter sections, each section providing some gain, serve to further amplify the tag signal and reject undesired signals. Typically these filters pass signals between 10× the excitation to at least 20× this frequency. Order of the filters is commonly between 4 and 8. Net gain through this block at mid frequencies is expected to be on the order of 60 dB.

### Analog to Digital Converter

After conditioning by the gain and filtering blocks just described, the tag signal is then converted to the digital domain by a 16 bit Analog to Digital converter which runs at some multiple of the excitation frequency. Common multiples are 64 and 128 in EAS systems and it is expected that will be the case here. Alternate selectable inputs to the A to D converter will be provided to allow the system to monitor other analog functions such as the excitation coil voltage and current, etc. . . .

### System Controller Tag Signal Processing

While the system controller performs other housekeeping tasks, it s primary function is to further process the digitized tag signal to enhance the tag response and reject all others, and then make decisions as to the nature of the signal and if declared a tag, sound the beeper and light a detection LED.

The most fundamental part of this process is a technique known as synchronous averaging which takes advantage of the fact that the same processor is acting as a source for the excitation and a processor of the resulting response. The result of this scenario is that all tag response signals must be exactly synchronous with the excitation signal. Communication theory always finds that synchronous demodulation yields the best recovered signal in the presence of noise and other interference. With high order synchronous averaging very good rejection of non tag signals is provided and the subsequent processes then analyze very clean response.

Following this linear filtering process a number of additional processes extract further parameters from the tag signal. Key among them is an Fourier Transform which extracts frequency distribution information and phase information. Other processes look at time gated energy, rate of change of energy, and balance of odd and even harmonic energy.

All of this data is fed into a rules based process which determines whether an object is a tag or not by examining all of this information together. if a tag is determined to be present, the appropriate annunciators are energized to inform the user that a tagged sponge has been located in the detection zone of the wand.

Finally the system controller itself may be either a classic DSP device optimized for signal processing tasks such as the synchronous filtering, or as is becoming more popular a single chip nigh speed general purpose microcontroller. Examples of suitable DSP s include the ADSP-21XX family from Analog Devices Inc. Similarly a suitable microcontroller might be the Atmel A9OSAM7SXXX family of ARUVI core based devices.

### Power Management

Unlike the EAS systems that have used this technology in the past, the v/and intended for use here will be battery operated to eliminate a cumbersome cable which might interfere in the scanning of the patient. Cables also pose reliability and cleaning challenges in these environments.

Fortunately a combination of two factors make battery operation of such a want increasingly practical. First there is the use of high efficiency class D amplifiers. These devices allow production of high power audio band signals with efficiencies over 80%. Our power source therefore need only supply 1.2 times the desired 20 W or 24 W to the amplifier. Previously class AB type amplifiers were generally not more than about 50% efficient and thus a 40 W power supply would be required in the same application. The next key enabler is the availability of high discharge rate Lithium-Ion Batteries. Prior to this development a device such as this which requires high levels of power for short periods of time would have been equipped with NiMH (Nickel Metal Hydride) cells. These are still today the dominant cells in cordless drills and other applications which require very high instantaneous power. Unfortunately these are heavy cells, a characteristic that is undesired.

Recently several companies, key among them Kokam of Korea, have introduced very high discharge rate lithium cells. These offer a combination of the high discharge rate of NiMT-I cells and the high energy density, lowweight of traditional Li-Ion cells. A series stack of 3 or 4 of these cells comprise the battery pack.

Besides the battery we have an integrated fast charger that will charge the Li-Ion pack from an AC line operated supply when the unit is in its wall mounted cradle. LED s will be provided to indicate state of charge. A charge time of less than 30 minutes is planned which will provide a continuous run time of approximately 5 minutes.

Raw cell voltage will be used to power the excitation amplifier as shown for maximum efficiency. On board switch mode regulators will convert the high pack voltage clown to low voltages required to run the balance of the system analog and digital circuitry.

As shown in FIG. 1, a laparotomy sponge 1 can include a plastic ribbon 3 that includes a magnetic amorphous wire 2. The magnetic amorphous wire 2 allows detection of the device by a detector. The plastic ribbon 3 would typically be designed to be x-ray opaque to allow detection by x-ray.

In creating the plastic ribbon 3 with a magnetic amorphous wire 2 embedded within it, an extrusion process may be used, as shown in FIG. 5. FIG. 2 shows a detailed view of an embodiment of the embedded result.

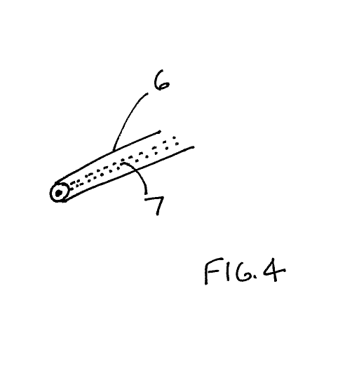

Other surgical tools and equipment may also be used to incorporate the magnetic amorphous wire 2 to allow detection. As shown in FIG. 4, the magnetic amorphous wire 7 may be encased inside a plastic coating to form a plastic sting 6. The plastic string 6 may be woven into a gauze sponge 5, as shown in FIG. 3. The resulting gauze sponge 5 of the invention could then be detected by a scan with a detector.

It will be readily understood by those persons skilled in the art, that the present invention is susceptible to broad utility and application in detecting surgical sponges and equipment. Many embodiments and adaptations of the present invention, other than those described, as well as many variations, modifications, and equivalent arrangements, will be apparent from or reasonably suggested by the present invention and foregoing description thereof, without departing from the substance or scope of the invention.

While the foregoing description illustrates and describes exemplary embodiments of this invention, it is to be understood that the invention is not limited to the construction and design disclosed herein. The invention can be embodied in other specific forms without departing from the true invention.

We claim:

1. A device for use in surgical procedures comprising:
an elongated magnetic amorphous metal wire for detection.

2. A surgical sponge comprising:
a magnetic amorphous metal wire for detection.

3. The surgical sponge according to claim 2, further comprising an extruded polyvinyl chloride barium sulfate ribbon that allows x-ray detection of the ribbon.

4. A system of detecting surgical accessories comprising:
an elongated magnetic amorphous metal wire attached to the surgical accessories,
a detector wherein the detector may be placed in proximity to the surgery and an alert will notify a user of the presence of the surgical accessories.

5. The system of detecting surgical accessories according to claim 4, wherein the detector is a hand-held scanner.

6. The system of detecting surgical accessories according to claim 5, wherein the detector is battery powered and capable of being surgically sterile.
